(19) United States
(12) Patent Application Publication
(10) Pub. No.: US 2006/0264775 A1
(43) Pub. Date: Nov. 23, 2006

(54) Methods of and apparatus for determining fluid volume presence in mammalian tissue

(76) Inventors: Gary N. Mills, Gladstone, OR (US); Philip D. Benz, Portland, OR (US); Herbert J. Semler, Portland, OR (US)

Correspondence Address: Stoel Rives, 900 SW Fifth Avenue, Suite 2600, Portland, OR, 97204-1268, US

(21) Appl. No.: 10/549,330

(22) PCT Filed: Mar. 11, 2004
(86) PCT No.: PCT/US04/07508

§ 371 Date: Sep. 12, 2005

Related U.S. Application Data
(60) Provisional application No. 60/454,846, filed on Mar. 14, 2003.

(51) Int. Cl. A61B 5/05; A61B 5/04

(52) U.S. Cl. 600/547; 600/509

(57) ABSTRACT

Methods and apparatus (20 ) process noninvasively measured electrical bio-impedence values and perform a technique that indicates whether there exists a change from a homeostatic fluid condition, preferably with respect to blood loss, in mammalian tissue. Analyses can be performed to determine a presence or a change in volume of fluid in an anatomical space of a mammal. A preferred implementation of such technique is embodied in an instrument that carries out a method that may predict an onset of a hemorrhagic shock condition.

### TECHNICAL FIELD

The present invention relates to determining the presence of a volume of fluid and, in particular, to methods and apparatus that process noninvasive electrical bio-impedance measurements to determine the presence of fluid volume in mammalian tissue.

### BACKGROUND OF THE INVENTION

Bio-electrical impedance or electrical bio-impedance is a complex quantity that in biological-electrical context represents the ratio of electrical current applied to and a resulting voltage measured across living biological tissue. The measured voltage as a function of applied frequency has amplitude and phase or real and imaginary components. Electrical bio-impedance has been used in several clinical applications, including evaluations of body composition, including both body fats and fluids, and of various hemodynamic or cardio-respiratory measurements. Heethaar et al. U.S. Pat. No. 6,339,722 describes an apparatus utilizing bio-impedance at multiple frequencies for the purpose of measuring body fluids, including various hemodynamic and cardiac measurements, and the distribution between extracellular and intracellular fluid components. Withers et al. U.S. Pat. No. 5,280,429 also describes a multiple frequency bio-impedance method and apparatus for fluid-monitoring purposes to determine various aspects of body composition, including intracellular and extracellular body fluid components. Liedtke U.S. Pat. No. 6,631,292 describes a device for measuring the resistance and reactance of a subject or segment thereof for the purpose of accurately providing a body composition measurement with a low level of noise caused by isolation of the subject from the electronic circuitry of the device. Yoshida U.S. Pat. No. 6,590,166 describes an apparatus similar in feature and appearance to a weight-measuring scale that further includes electrodes for use in making bio-impedance measurements to determine body fat.

Several methods and devices have been described that incorporate bio-impedance for the purpose of measuring hemodynamic or cardio-respiratory parameters. Porat U.S. Pat. No. 6,277,078 describes a system and method that monitor parameters associated with heart function, including intra-cardiac and intravascular pressures. The system requires at least two sensors implanted in the heart and in a blood vessel, as well as an implanted device in communication with the sensors. Baura et al. U.S. Pat. No. 6,561,986 describes an apparatus and a method using impedance and ECG (electrocardiographic) waveforms that are analyzed using discrete wavelet transforms to assess hemodynamic parameters, including cardiac output, in an organism. Baura et al. U.S. Pat. No. 6,636,754 describes, in conjunction with a specific electrode patch, an apparatus and a method using bio-impedance detected through the thoracic cavity of a living subject to determine the subject's cardiac output. Hepp et al. U.S. Pat. No. 6,602,201 also describes an apparatus and a method for determining cardiac output.

Electrical bio-impedance may also be used in impedance tomography, an imaging technique in which images of conductivity within a cross-sectional plane of a subject's body may be made from data collected by the use of an array of stimulation and measurement electrodes placed around the periphery of the body or part thereof to be evaluated. Barber et al. U.S. Pat. No. 5,626,146 describes an apparatus designed to improve the quality and reliability of images collected through the impedance tomography method by varying the time periods in which the measurements are made. Cherepenin U.S. Pat. No. 6,236,886 describes a method implemented to obtain impedance tomography cross-sectional images of conductivity with a signal-to-noise ratio higher than that accomplished by the prior art.

Electrical bio-impedance has been in clinical use for many years for body composition and hemodynamic measurement applications. Recent examples of instruments performing such measurements are those made and sold by CardioDynamics International Corporation (assignee of U.S. Pat. Nos. 6,636,754; 6,602,201; and 6,561,986) and Xitron Technologies (assignee of U.S. Pat. No. 5,280,429).

The prior art and the literature do not, however, discuss application of electrical bio-impedance to the diagnosis and monitoring of subjects at risk of going into the clinical state of shock. Shock occurs when a mammalian body has become unable to perfuse itself, thereby creating a condition in which blood pressure and cardiac output drop, resulting in a downward spiral involving end organ damage caused by hypoxia and ultimately resolving in death of the subject. In the case of septic shock, shifts in fluid between extracellular and intracellular spaces and shifts in total body water may cause a cascade resulting ultimately in an onset of shock. Shock can also be caused by loss of fluid from body components. In the case of hemorrhagic shock, which is defined as a dire physiological result of a reduction in circulating blood volume such as from a trauma-related event, blood lost from the circulatory system reduces fluid volume required for adequate perfusion. Hemorrhagic shock can occur whether the blood is lost outside of the body or whether the blood is lost inside of the body but still outside of the circulatory system.

Internal hemorrhage, frequently caused by blunt trauma, is difficult to detect and is often unaccompanied by clinically significant signs on the body surface that would be indicative of such internal injury. If left undetected and untreated, uncontrolled internal hemorrhaging can lead to shock, irreversible injury, and death. Examples of sources of such internal hemorrhage include liver or splenic contusions or lacerations often encountered in motor vehicle accidents, in which there are no outward signs of injury, such as penetrating wounds or bruising. In the military venue, further examples include impact of high-velocity projectiles on body armor and shock waves from explosive blast. In these examples, undetected bleeding continues into the body cavity for an extended period, depleting the circulatory system and thereby causing hypovolemia which, if left uncontrolled, leads to shock.

There are currently no field-deployable tools that can be used to detect presence of internal bleeding, particularly while there is such bleeding at an early, pre-shock stage. A significant need exists for a detection device that provides early information regarding internal hemorrhage in both military and civilian emergency medicine and trauma sectors.

Non-invasive blood pressure (NIBP) is commonly used to detect onset of shock; however, blood pressure drop is a late indicator and may be useful only to monitor shock caused by internal hemorrhage. Circumstances in which a patient is suspected of having internal bleeding permit a physician to order a computed tomography (CT) scan or magnetic resonance imaging (MRI) to definitively image presence and location of bleeding. Such circumstances necessitate arrival of the patient at a hospital. However, these procedures are expensive and can prolong the time required for the patient to receive definitive therapy. Moreover, because of their size and cost, CT scan and MRI devices are not normally present in the field, i.e., in an ambulance or with a first-responder.

Diagnostic peritoneal lavage (DPL) is a commonly used invasive method in the hospital emergency room, where a catheter is used to drain fluid from the abdomen, sometimes requiring infusion of saline. Fluid removed by DPL is then analyzed by the hospital laboratory to detect the presence of blood. Ultrasound imaging using the FAST (focused abdominal sonogram for trauma) method is also sometimes used in the hospital emergency room to detect internal bleeding. However, the instrument user interface makes this technique operator-dependent, and therefore subjective, and requires specific training and skill in the use of ultrasonography. With the exception of NIBP, all of these diagnostic modalities require the patient to be in the hospital and necessarily extend the time to definitive therapy.

What is needed are methods and apparatus that can non-invasively detect fluid shifts which could cause onset of shock. More particularly, methods and apparatus are needed that non-invasively measure electrical bio-impedance values and perform a technique that indicates the nature of a change, if any, from a homeostatic fluid condition in mammalian tissue. A preferred implementation of such methods, apparatus, and technique would be a test to predict, assess, or monitor a hemorrhagic shock condition, in which such hemorrhaging occurs internally, inside the body.

### SUMMARY OF THE INVENTION

The method of the present invention in its most general aspect determines shifts in fluids in mammalian tissue. The method entails positioning members of a set of injection electrodes and members of a set of measurement electrodes at known locations on the surface of the body of a mammal. The injection electrodes introduce electrical current flow through the body tissue. Electrical current flow paths established by the injection electrodes define injection vectors generally along which electrical currents flow between two or more of the injection electrodes. The measurement electrodes define measurement vectors along which electrical voltages are measured as a result of the electrical currents flowing between the injection electrodes. The injection and measurement vectors collectively define an anatomical space of the body tissue.

The injection and measurement electrodes are connected to an electrical bio-impedance measurement instrument that derives information indicative of the electrical bio-impedance of the anatomical space. An electrical bio-impedance value is derived from the signals for each one of the vectors. Each value derived characterizes the electrical bio-impedance of an associated region of the anatomical space of body tissue. The electrical bio-impedance values are analyzed to detect shifts of fluids in the anatomical space of body tissue. Analyses performed on the bio-impedance values can determine the nature of such shifts in fluids. Examples of such fluid shifts generally include distribution between extracellular fluid (ECF) and intracellular fluid (ICF), changes in total body water (TBW), changes in hemodynamic and cardio-respiratory parameters including cardiac output, presence of fluid accumulations inside the body, and extent of bleeding out of the circulatory system into the body or out of the body.

A preferred embodiment of the method is implemented in a noninvasive internal hemorrhage detecting instrument that determines whether there is a depletion or an increase of blood in an internal region of human body tissue either inside or outside of the circulatory system. The internal hemorrhage detecting instrument uses electrical bio-impedance, measured in response to a current injected into the body, to detect presence of internal bleeding in the body. Electrical bio-impedance measurements use alternating current measured at either a single frequency or at multiple frequencies. The instrument measures the general presence and extent of internal bleeding in body tissue by analyzing changes in electrical bio-impedance, which is influenced by the amount of fluid in the tissue. More particularly, the instrument can detect changes in the extent of accumulations of blood within the body tissue and outside of the circulatory system. Variations of the preferred embodiment may permit detection and evaluation of accumulations of fluids other than blood within the body, for example, pleural exudate, plasma, mucus, or infused fluids. The instrument is generally intended for use in trauma, emergency, critical care, and other medical situations.

While other applications of electrical bio-impedance evaluate changes in fluids or specifically evaluate changes in various hemodynamic or cardio-respiratory parameters, this preferred embodiment evaluates the general presence and changes in accumulations of fluids inside the body, more particularly for situations in which such fluids include blood outside the circulatory system.

Changes in electrical bio-impedance, which are determined by using an injected alternating electrical current, are shown to correlate with changes in tissue fluids, which are correlated with changes in volume of fluid contained inside the body but outside the circulatory system. A discrete electrical bio-impedance value is derived from the signals for each vector defined by the different pairs of the injection and measurement electrodes. Each derived value characterizes the electrical bio-impedance of an associated region of the anatomical space of body tissue. The relative vector-specific changes in electrical bio-impedance values collected over time are analyzed to determine the general location and extent of fluid accumulation or loss. The electrical bio-impedance data collected over time are analyzed and used to determine trends and measure cumulative and instantaneous parameters of blood loss, which are referred to herein as blood loss indices (BLI). The BLI are functions of electrical bio-impedance changes and rates of change.

A preferred use of the instrument is by first responders or paramedics, who would apply the instrument to patients before or during their transport to a hospital emergency room. Patient data are provided to emergency department doctors while the patent is en route to or upon the patient's arrival at the emergency room. A benefit of the instrument is early access by physicians to information about internal bleeding, thereby enabling quick guidance for clinical decision making.

Additional embodiments may include different numbers, locations, and configurations of electrodes placed on the body; different combinations of electrical current and frequency; and different processing or analyses to provide additional features. Such features include evaluating changes in extracellular and intracellular fluids; evaluating changes in total body water; evaluating changes in hemodynamic and cardio-respiratory parameters, including cardiac output and cardiac stroke volume; evaluating an inflammatory state in a body; evaluating changes in extracellular fluids, in addition to blood; acquisition and processing of electrocardiograms; detection of electric pulse-generator pulses; and determination of respiration. Detector apparatus may further be included together with other medical diagnostic and therapeutic devices, as later described. These embodiments all contribute to the utility of the invention in a trauma, a critical care, or an emergency medical environment. This is true whether the invention is deployed in the hospital or in the field, for example, at the scene of an accident.

Additional objects and advantages of the invention will be apparent from the detailed description of preferred embodiments thereof, which proceeds with reference to the accompanying drawing.

### BRIEF DESCRIPTION OF THE DRAWINGS

FIGS. 1A and 1B are respective top and bottom plan views of a multiple contact electrode assembly formed on a patient skin patch.

FIG. 2 is a block diagram of a fluid volume detector instrument of the present invention.

FIG. 3 is a diagram showing an anterior view of the locations of electrode placement and associated current injection and measurement vectors on the torso of a subject undergoing a fluid loss or accumulation study verifying the method of the present invention.

FIG. 4 is a diagram showing an anterior view of a detector instrument attached to a mammalian body by means of lead wires and electrodes.

FIG. 5 is a diagram showing the electrode configuration described in a preferred embodiment used to acquire electrocardiograms.

FIG. 6 is a block diagram of a circuit used to acquire an electrocardiogram and to detect the presence of implanted electrical pulse generator signals.

FIG. 7 is a diagram showing alternative locations of electrodes on a posterior view of a mammalian body.

FIGS. 8A and 8B are diagrams showing combinations of the detector instrument with other medical diagnostic and therapeutic equipment.

### DETAILED DESCRIPTION OF PREFERRED EMBODIMENTS

To practice a preferred embodiment of the invention directed to evaluating internal hemorrhage, a medical practitioner uses at least one pair of injection electrodes to inject electrical current into the body and another pair, or more, of measurement electrodes to measure electrical voltage produced as a result of electrical current flowing through the body tissue. Each measurement electrode is positioned in proximity to an injection electrode. The electrodes are made of electrically conductive material, preferably Ag—AgCl with an electrically conductive gel to couple to the body surface. An electrode “patch” may contain at least two electrically active elements or electrodes, one of which injects electrical current and the other one or other ones of which measure the resulting voltage, in association with other current injection and voltage measurement electrodes.

Injected current is in a range of between 50 μA rms and 500 μA rms, at a voltage of not greater than about 20 volts rms. These pairs of electrodes, which may be contained on a single nonconductive backing material, are independently wired to the detector instrument by electrically conductive cables for injecting electrical current into one electrode and measuring voltage with the other electrode. Multiple pairs of electrodes are used and placed on the subject body surface. The electrodes are connected to the detector instrument by electrically conductive cable, and the conductive gel connects the electrode to the surface of the body. The substrate or “backing” of the electrode patch has an adhesive to secure it to the body surface. If more than one electrode is contained on a substrate, the electrodes are electrically isolated from the other electrode or electrodes. Alternatively, an electrically conductive adhesive may be used as the gel to electrically connect and adhere the electrode to the body surface.

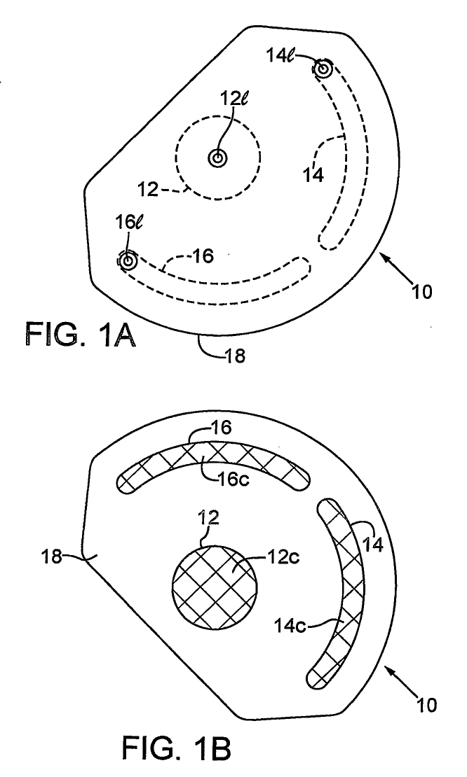

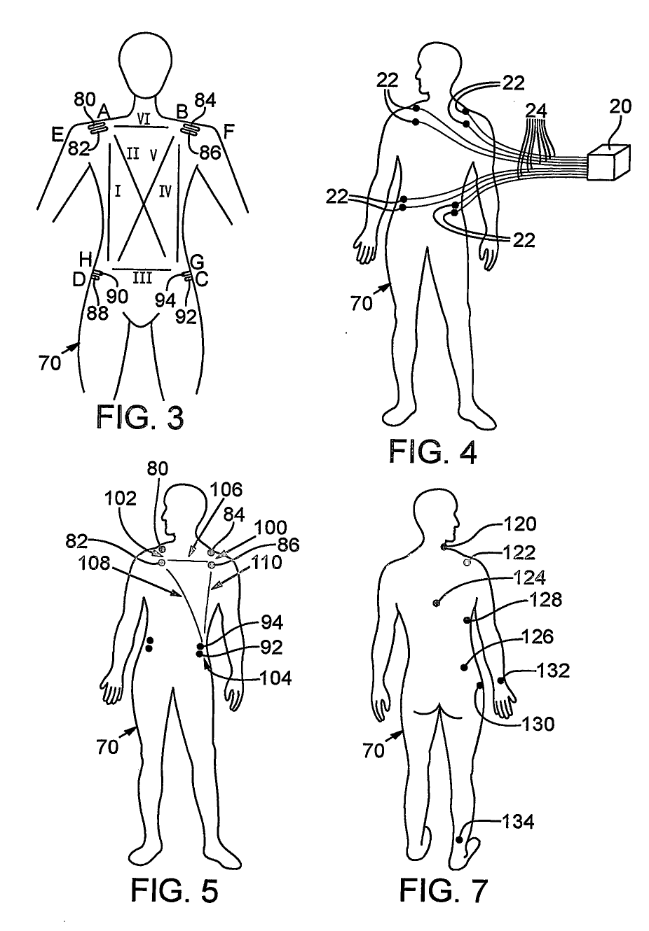

FIGS. 1A and 1B are respective top and bottom views of an exemplary multiple electrode assembly 10 formed on a patch that can be applied on the skin of a patient. Electrode assembly 10 includes a circular electrode 12 positioned medially of two circular segment electrodes 14 and 16. Electrodes 12, 14, and 16 are supported on a substrate 18. FIG. 1A shows lead wire connection points 12l, 14l, and 16l for electrodes 12, 14, and 16, respectively. FIG. 1B shows active electrode conductive contact areas 12c, 14c, and 16c of electrodes 12, 14, and 16, respectively. One or more of electrodes 12, 14, and 16 can be used for electrical current injection or voltage sensing. For example, electrical current could be injected through circular electrode 12 and voltage measurements taken from one or both of segment electrodes 14 and 16. In one embodiment of electrode assembly 10, circular electrode 12 constitutes the injection electrode and circular segment electrodes 14 and 16 constitute the measurement electrodes. The use of electrode assembly 10 would replace the separate injection and measurement electrode patches placed in proximity to each other at the locations defined as the terminal points of the vectors, as generally shown in FIG. 3.

The electrical current is injected at a single frequency or multiple frequencies, either sequentially or simultaneously. The number of discrete frequencies at which current is injected may be as many as 100. The range of frequencies is from 1 kHz to 500 kHz. Electrical current is preferably injected with between one and twelve frequencies in the range 5 kHz to 300 kHz. Multiple frequencies are used to more accurately distinguish extracellular fluid changes as compared with changes measured at only a single frequency. The differences in electrical bio-impedance at different frequencies relate to changes in volume of intracellular fluid versus extracellular fluid. Sequential or simultaneous current injection and sensing between different pairs of electrodes may be used to achieve multiple measurement vectors to determine the presence and general location of internal bleeding. A measurement is a single collection of readings for all frequencies and all vectors. The frequency of taking measurements ranges between ten each second to once each 60 seconds for purposes of calculating the blood loss index. The vectors are designated as current paths or measurement paths between pairs of electrodes placed on the body surface.

An alternative to the use of discrete frequencies in the injection of electrical current is the use of a range of continuously varying frequencies of applied electrical current or other complex current waveforms.

Electrode placement is made in regions defined by anatomical landmarks that are easily recognizable by intended users. The landmarks are preferably bilaterally in the regions defined by the deltoid, pectoralis, and trapezius muscles for two of the electrode pairs, and bilaterally in the regions defined by the lower rectus abdominus and gluteus medius muscles for the other two electrode pairs.

The detector instrument is an electronic system that is capable of performing several basic tasks including repeatedly injecting current into the subject body, repeatedly sensing voltages or currents resulting from the injected current, and repeatedly calculating electrical bio-impedance from the sensed voltages or currents. The detector may be either line-powered or battery-powered.

Additional features of the detector instrument include storage and output of data, which may include instrument settings, status, sensed voltage and current levels, and impedances. Additional optional features include a display, at least one communication link in which the detector instrument is able to export data using either wire line or wireless media to a location external to the instrument, and systems for collecting, storing, delivering, or displaying other vital signs information (for example, electrocardiogram, blood pressure, hemodynamics including cardiac output, or pulse oximetry).

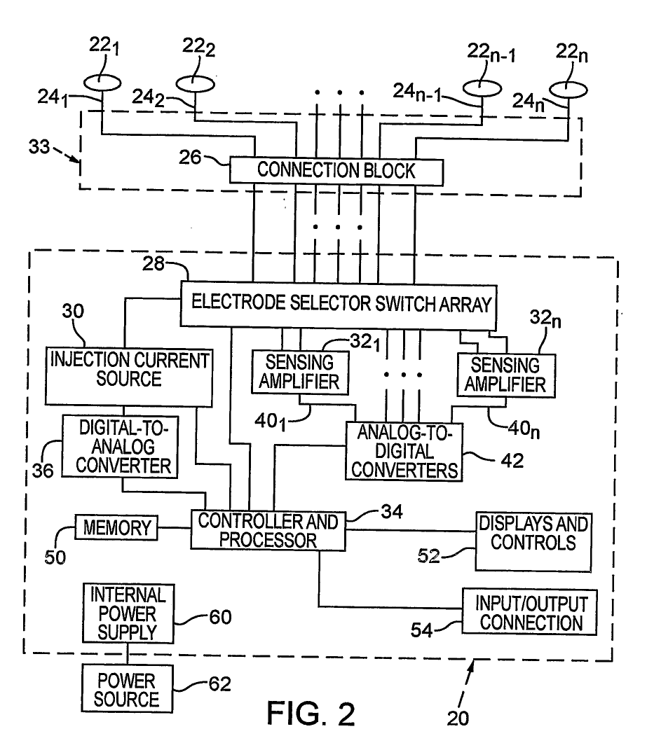

FIG. 2 is a block diagram of a fluid volume detector instrument 20 configured in accordance with the invention to detect an incidence of blood loss. A number, n, of electrodes 22 are connected to detector instrument 20 by respective independent electrically conductive cable leads 24 terminating in a connection block 26. Connection block 26 may be located inside or outside of the housing of detector instrument 20, as described below. Injection and measurement electrodes 22 may be set on a common piece of nonconductive backing material (FIGS. 1A and 1B). Some form of electrical high-power protection may optionally be included in line with leads 24 to prevent electrical energy from a defibrillator shock, or other high energy discharge, from damaging the electronic circuits of detector instrument 20. Connection block 26 connects electrodes 22 by leads 24 to electrode switch array circuitry 28 that controls which ones of electrodes 22 inject electrical current and measure voltage. Switch array circuitry 28 also conducts injected current produced by an injection current source 30 to the injection electrodes 22 and conducts to analog sensing amplifier circuitry 32 electrical voltages, including those produced in response to the electrical currents flowing between the injection electrodes 22 and developed across the measurement electrodes 22. The broken line rectangular box enclosing connection block 26 indicates a connection block module 33 that is releasably attachable for matable connection by a housing connector of detection instrument 20 to electrode selector switch array circuitry 28. The multiple electrodes 22 are connected by associated leads 24 to connection block module 33. Connection block module 33 may optionally include one or more of a battery, defibrillator discharge protection, or memory.

An input of electrode switch array circuitry 28 connects to injection current source 30, which generates the electrical current to be injected through the injection electrodes. Outputs of switch array circuitry 28 connect to sensing amplifier circuitry 32, which amplifies the voltages developed across the measurement electrodes. Microcontroller and microprocessor circuitry 34 (hereafter, processor circuitry 34) is programmed with instructions that control electrode switch array selection of electrodes 22 for current injection and voltage sensing. Electrode selector switch array circuitry 28 is configured for independent selection of the multiple electrodes 22 in response to control command information delivered from processor circuitry 34. Injection current source 30 is of a programmable type and is connected to a digital-to-analog converter (DAC) 36, which converts current injection instructions into alternating current of proper frequency and amount, and sends back to processor circuitry 34 an indication of the execution of the instruction for the injection current frequency and amount. DAC 36 is connected to processor circuitry 34, which stores, modifies, and issues instructions for current injection and which receives actual frequency and amount data for electrical current injection. Sensing amplifier circuitry 32 preferably includes separate sensing amplifiers having outputs that are connected to inputs 40 of analog-to-digital converter (ADC) circuitry 42, which converts the analog voltages to digital data. ADC 42, which is composed of multiple converter circuits or a single converter circuit with selectable inputs, is connected to processor circuitry 34 for digital signal processing (DSP) of the sensed voltages. Digital signal processing is conducted and calculations made from the processed signals, resulting in information that indicates whether there exists internal blood loss. More specifically, for the preferred embodiment, processor circuitry 34 is programmed with instructions to carry out three data acquisition and analysis functions. First, processor circuitry 34 processes signals representing the injection electrical currents and the produced electrical voltages corresponding to different pairs of the injection and measurement vectors. Second, processor circuitry 34 computes from each of different pairs of the injection and measurement vectors an electrical bio-impedance value that characterizes the electrical bio-impedance of a region of mammalian tissue in an anatomical space. Third, processor circuitry 34 analyzes the electrical bio-impedance values to detect a presence of or a change in a volume of fluid in the anatomical space.

Processor circuitry 34 can be implemented as one or more microcontroller devices, one or more microprocessors, or application specific integrated circuitry (ASIC) combining their functions. Moreover, other of the functions of the electronic components of detector instrument 20 can be combined into an integrated circuit (IC), e.g., DAC 36, ADC 42, and sensing amplifiers 32.

Memory stores 50 connected to processor circuitry 34 are used to store instructions and data for actual current injection frequency and amount, data for sensed voltages, and information that indicates whether there exists internal blood loss. Displays and controls 52 are connected to processor circuitry 34. The displays may visually present data for actual current injection frequency and amount, data for the sensed voltages, and other information, including BLI. The displays may also present user feedback based on inputs from controls. More preferably, the data may be presented textually and graphically. The controls are used for controlling operational functions of detector instrument 20. An input/output connection device 54 connected to processor circuitry 34 provides a capability of receiving data or instructions or of exporting contents of memory stores 50 or real time data to a location external to detector instrument 20. All or a portion of memory stores 50 may optionally be removable from detector instrument 20 and, upon removal, be capable of retaining stored computed electrical bio-impedance values to facilitate interchangeability between multiple detector instruments 20 or other external devices, e.g., a computer, that are separate from detector instrument 20.

An internal power supply 60 provides electrical power to detector instrument 20 to enable it to operate. Internal power supply 60 receives power from a power source 62, which may be in the form of batteries or a DC converter converting power from mains or other source of power. Mains isolation is included in the electronic circuitry for purposes of electrical safety. Batteries may be included in the detector instrument 20 or combined into an assembly that includes at least one of cable leads 24, electrodes 22, at least a portion of memory stores 50, electrical high-power protection circuitry, and a connector to provide securement and electrical connection to the detector instrument 20. The electrical high-power protection circuitry is contained in the electronic circuitry of detector instrument 20 to protect against high-power electrical surges, such as, for example, from defibrillators.

The electrical bio-impedance values used to calculate blood loss indices include cumulative (as of initial measurements taken) values to generally correlate with cumulative internal blood loss, and instantaneous (as of the most current measurement) values to generally correlate with rate of internal blood loss. The trend of electrical bio-impedance values and calculated derivative data thereof are key indicators, as well as the current measurement compared to the trend or average of the data used to develop the calculated derivative data.

Features of detector instrument 20 include data storage, information display, and delivery of data. The data may include the settings and status of detector instrument 20, applied and sensed voltages, applied and sensed current levels, and electrical bio-impedances. The displays are capable of showing parameters, measurements, and blood loss index. Currents, voltages, and sensitivity are selectable under microprocessor control. Detector instrument 20 is preferably a portable, battery powered instrument. The inclusion of batteries as a power source 62 within detector instrument 20 facilitates its portability. It may also be line powered if an adaptor is used or if detector instrument 20 is made part of other equipment. Detector instrument 20 providing electrical bio-impedance information is combinable with other life signs devices (for example, electrocardiogram, blood pressure, hemodynamics including cardiac output, or pulse oximetry) and with defibrillators and pacemakers. Detector instrument 20 can communicate measurements, parameters, and blood loss index data to a location external to detector instrument 20 by wireline or wireless communication links, including short-range (e.g., BLUETOOTH) or long-range (e.g., cellular), either through onboard components or by an external connection (e.g., serial port interface) to external components.

FIG. 3 shows the locations of electrode placement and associated current injection and measurement vectors on the torso of a subject on whom the preferred embodiment has been practiced. These electrode placements and vectors are the same as those used in conducting the studies described elsewhere in this specification. A measurement electrode and an injection electrode are located generally at each of the four anatomical corners of the torso of a mammalian body 70. A right shoulder injection electrode 80 is generally located above the right clavicle on the trapezius muscle, and a right shoulder measurement electrode 82 is generally located below the right clavicle on the upper portion of the pectoralis muscle. A left shoulder injection electrode 84 is generally located above the left clavicle on the trapezius muscle, and a left shoulder measurement electrode 86 is generally located below the left clavicle on the upper portion of the pectoralis muscle. A right hip injection electrode 88 is generally located in the area near the upper portion of the right gluteus medius muscle or lower portion of the right external oblique muscle of the abdomen, and a right hip measurement electrode 90 is generally located above right hip injection electrode 88. A left hip injection electrode 92 is generally located in the area near the upper portion of the left gluteus medius muscle or lower portion of the left external oblique muscle of the abdomen, and a left hip measurement electrode 94 is generally located above the left hip injection electrode 92.

FIG. 3 also generally shows the locations of the current injection and measurement vectors used to acquire the bio-impedance data representative of the regions defined by the vectors of the anatomical space of body 70, described in the preferred embodiment. Vector I describes a region of the anatomical space between the right shoulder and right hip and includes right shoulder injection electrode 80, right hip injection electrode 88, right shoulder measurement electrode 82, and right hip measurement electrode 90. Vector II describes a region of the anatomical space between the right shoulder and left hip and includes right shoulder injection electrode 80, left hip injection electrode 92, right shoulder measurement electrode 82, and left hip measurement electrode 94. Vector III describes a region of the anatomical space between the right hip and left hip and includes right hip injection electrode 88, left hip injection electrode 92, right hip measurement electrode 90, and left hip measurement electrode 94. Vector IV describes a region of the anatomical space between the left shoulder and left hip and includes left shoulder injection electrode 84, left hip injection electrode 92, left shoulder measurement electrode 86, and left hip measurement electrode 94. Vector V describes a region of the anatomical space between the left shoulder and right hip and includes left shoulder injection electrode 84, right hip injection electrode 88, left shoulder measurement electrode 86, and right hip measurement electrode 90. Vector VI describes a region of the anatomical space between the left shoulder and right shoulder and includes left shoulder injection electrode 84, right shoulder injection electrode 80, left shoulder measurement electrode 86, and right shoulder measurement electrode 82.

The injection and measurement vectors described above portray the vectors as generally overlapping, e.g., measurement vector I overlaps injection vector I because injection electrodes 80 and 88 and measurement electrodes 82 and 90 defining the region of the anatomical space between the right shoulder and right hip are selected by electrode switch array circuitry 28 when a reading is taken. Alternatively, for a reading, a measurement vector and an injection vector may be generally non-overlapping, e.g., a reading may be taken when the injection electrodes selected by electrode switch array circuitry 28 are for vector I, with injection electrodes 80 and 88, and the measurement electrodes selected are for vector II, with measurement electrodes 82 and 94.

FIG. 4 shows detector instrument 20 connected to mammalian body 70 by electrodes 22 attached to cable leads 24. Mammalian body 70 is preferably in a generally supine position when detector instrument 20 is in use.

FIG. 5 shows the electrode configuration described in the preferred embodiment that may be used to additionally acquire electrocardiograms of various vectors or leads. A left arm electrode 100 may be the same as either left shoulder injection electrode 84 or left shoulder measurement electrode 86. A right arm electrode 102 may be the same as either right shoulder injection electrode 80 or right shoulder measurement electrode 82. A left leg electrode 104 may be the same as either left hip injection electrode 92 or left hip measurement electrode 94. These electrodes sense voltages and enable the acquisition, storage, and display of electrocardiograms (ECG) through their processing in an ECG circuit, which is well known to those skilled in the art. A Lead I ECG 106 is acquired using the vector between left arm electrode 100 and right arm electrode 102. A Lead II ECG 108 is acquired using the vector between right arm electrode 102 and left leg electrode 104. A Lead III ECG 110 is acquired using the vector between the left arm electrode 100 and the left leg electrode 104. Each of these ECG vectors additionally requires an indifferent ground electrode; an electrode not being used as a vector endpoint may be used as such. For example, left leg electrode 104 would be used as the indifferent ground electrode for the Lead I ECG 106. Additional ECG vectors may be mathematically derived from the voltages collected from left arm electrode 100, right arm electrode 102, and left leg electrode 104. Examples of these vectors include the augmented ECG leads, known as aVR, aVL, and aVF. Different configurations of electrodes enable the collection and derivation of additional ECG leads. The process of mathematically deriving the augmented ECG leads and using different configurations of electrodes to collect and derive additional ECG leads is well known to those skilled in the art.

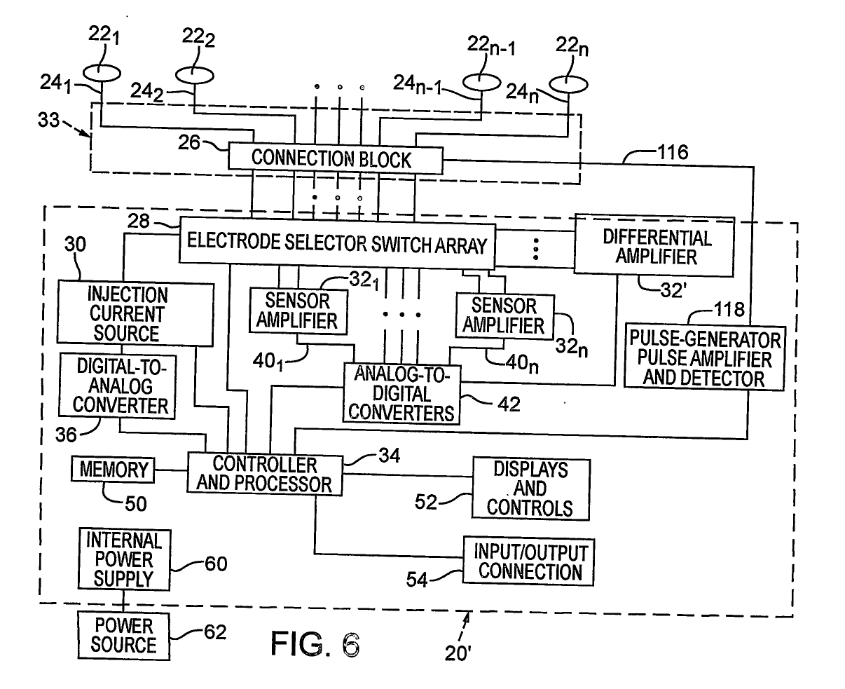

Circuitry required to acquire, store, and display the ECGs may be generally embodied within certain elements of detector instrument 20 shown in and described with reference to the block diagram of FIG. 2 and more particularly shown in FIG. 6 as modifications to the block diagram of FIG. 2. FIG. 6 shows that sensed voltages developed across electrodes 22 are conveyed by electrically conductive cable leads 24 terminating in connection block 26, in turn connected to electrode selector switch array circuitry 28. Electrode selector switch array circuitry 28 responds to command information from processor circuitry 34 to determine the electrode configurations described with reference to FIG. 5 for ECG signal acquisition and operation. The sensed voltages are conveyed to sensing amplifier circuitry 32, the outputs of which are connected to ADC 42 that convert the amplified sensed voltages into digital data. These digital data are sent to processor circuitry 34 for subsequent storage and display. At least one component amplifier of amplifier circuitry 32 is preferably a differential or instrumentation amplifier 32′ (hereafter differential amplifier 32′) that operates in a differential input mode. Differential amplifier 32′ has two or more input connections to the electrodes and an amplification gain suitable for ECG acquisition. Another aspect of ECG acquisition is the use of at least one electrically driven electrode to improve common mode noise rejection. A further aspect associated with acquiring, storing, and displaying ECGs is the detection of signals produced by an implanted electrical device used for cardiac, neural, or other tissue sensing or stimulation. Modified detector instrument 20′ includes a separate circuit that recognizes a pulse-generator pulse by its characteristic rapid voltage rise and fall, period of occurrence, or other waveform characteristic such as, for example, amplitude or phase. This circuit functions with a dedicated continuous connection 116 to at least one of the electrodes used for ECG acquisition or pulse-generator pulse detection.

Referring to FIG. 6, continuous connection 116 provides the acquired analog signal from at least one electrode 22 (e.g., electrode 22_{n,} in FIG. 6) used for ECG acquisition or pulse-generator pulse detection by an electrically conductive cable lead 24 (e.g., lead 24ₙ in FIG. 6) to connection block 26. Cable lead 24 is routed along two signal paths within connection block 26. The first signal path provides a dedicated continuous connection 116 to conduct the signal to pulse-generator pulse amplifier and detector circuitry 118, the output of which connects directly to processor circuitry 34. The second signal path runs through connection block 26 to enable selection and switching by electrode selector switch array circuitry 28, as with the other electrodes 22. Pulse-generator pulse amplifier and detector circuitry 118 amplifies the incoming analog voltage signal, processes the signal through a high-pass filter to exclude voltage excursions too low in amplitude to comprise a pulse-generator pulse, and compares the remaining voltage excursions to ranges of values for pulse width, amplitude, slope of the voltage excursion over time, period, and phase or polarity to determine valid pulse-generator pulses. This process is well-known to those skilled in the art. In an alternative preferred embodiment, Lead I ECG 106, Lead II ECG 108, and Lead III ECG 110 are routed through switch array circuitry 28 to separate sensor amplifiers 32′ operating in a differential input mode. Switch array circuitry 28 responds to command information from processor circuitry 34 to select which ones of electrodes 22 form leads 106, 108, and 110. Preferably a pair of leads 24 is provided as a continuous connection 116 to pulse-generator pulse amplifier and detector circuitry 118, which produces an output that represents the signal characteristics of a pulse produced by a pulse generator (e.g., a pacemaker) implanted in a subject mammal. Processor circuitry 34 is programmed with instructions to process signals corresponding to those appearing at the outputs of the sensor amplifiers 32′ and optionally the output of pulse-generator pulse amplifier and detector circuitry 118 to produce an ECG signal representation that includes an indicator of a pulse-generator pulse. The ECG signal may be presented as a display image. If it is displayed, the ECG signal may be presented with or without a pulse-generator pulse indicator component.

FIG. 7 shows examples of additional locations for placement of current injection and measurement electrodes on mammalian body 70. Neck locations 120 may be used, with electrodes placed on one or more sides, as is sometimes done with bio-impedance monitors evaluating hemodynamic, cardio-thoracic, or body composition parameters. Upper back locations 122, on one or both sides of or on the medial line, may also be used. Middle back locations 124 or lower back or gluteus maximus locations 126, on one or both sides of or on the medial line may also be used. Mid-torso locations 128, generally in the axillary line, may also be used on one or both sides as is sometimes done with bio-impedance monitors evaluating hemodynamic, cardio-thoracic, or body composition parameters. Hip locations 130 may also be used on one or more sides, where the electrodes are placed generally in the area of the lateral aspects of the head of the femur. Arm locations 132 may also be used on one arm or both arms, where the electrodes are placed generally on the arm or more particularly the lower arm, wrist, or hand area. Leg locations 134 may also be used on one leg or both legs, where the electrodes are placed generally on the leg or more particularly the lower leg, ankle, or foot area.

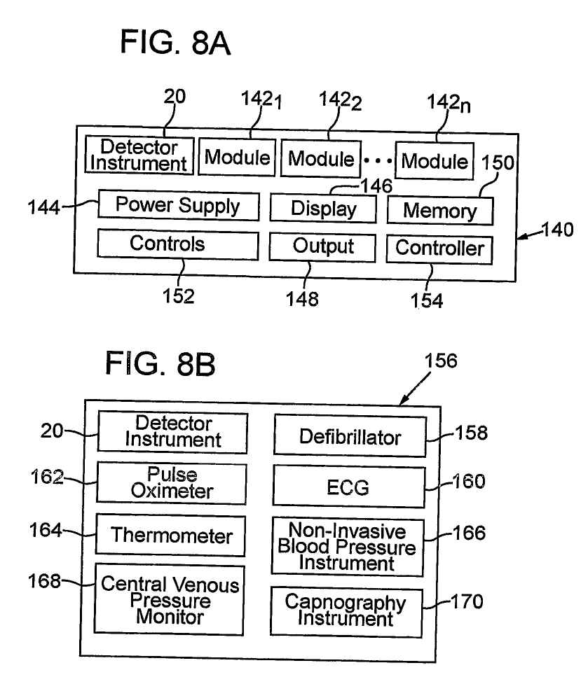

FIGS. 8A and 8B are block diagrams showing combinations of detector instrument 20 with other medical diagnostic and therapeutic equipment. FIG. 8A shows detector instrument 20 generally contained in an enclosure 140 in which at least one of additional, optional separate plug-in or built-in medical diagnostic or therapeutic modules 142 are housed as an integrated system. Examples of modules 142 include ECG, pulse oximeter, non-invasive blood pressure, thermometer, capnography, respiration, non-invasive cardiac output, central venous pressure monitor, plethysmography, pneumography, and cardiac defibrillator. Enclosure 140 may further include at least one of a power supply 144, a display device 146, for example, an LCD panel; an output device 148, for example a Universal Serial Bus (USB) or serial or local area network connector or wireless transmitter; memory 150 for retaining instructions and data sourced from modules 142; controls 152 and controller subsystem 154 for common operational access by one or more of modules 142 contained within enclosure 140. FIG. 8B shows detector instrument 20 contained in an enclosure 156 that also contains as a non-integrated collection of independently operating diagnostic or therapeutic equipment modules. Examples of such equipment modules include a cardiac defibrillator 158, an ECG acquisition device 160, a pulse oximeter 162, a thermometer 164, a non-invasive blood pressure instrument 166, a central venous pressure monitor 168, or a capnography instrument 170 as shown. Other examples of such equipment modules include a noninvasive cardiac output measurement instrument, plethysmography instrument, or pneumography instrument. Skilled persons will appreciate that these combinations are only representative examples of the scope of possible combinations of medical diagnostic and therapeutic devices.

### EXAMPLE 1

The following example is taken from a ten-person study in which the method of the invention was carried out to detect fluid accumulation or loss in the torso of a human being. Each of ten volunteers first voided his bladder, rested in a supine position, and received electrodes placed in various combinations on the right and left shoulders and on the right and left hips. The shoulders and hips enabled study of the torso space and provided prominent landmarks that allowed replication of the experiment. A current-injection electrode and a voltage-measurement electrode were placed at each shoulder and hip location, as shown in FIG. 3. The electrodes were connected to a Xitron 4200 electrical bio-impedance measurement instrument, which was implemented with HYDRA acquisition software to determine intracellular and extracellular volume within a defined space. The data set derived from use of the Xitron 4200 instrument is based on multiple frequency (e.g., Fourier) analysis and data reduction.

The electrodes were placed on the volunteer to define six vectors, and electrical bio-impedance values were acquired for each vector. The vectors included right shoulder-right hip (I), right shoulder-left hip (II), right hip-left hip (III), left shoulder-left hip (IV), left shoulder-right hip (V), and right shoulder-left shoulder (VI). FIG. 3 shows the locations of electrode placement and associated current injection and measurement vectors on the torso of the volunteer. Each electrode represents a single electrical contact functioning as a current source, current sink, or voltage sensing element. Table 1 lists the electrode lead pairs (identified by Arabic numerals) used to inject electrical current and sense voltage for each vector (identified by Roman numerals).

TABLE 1

| Vector | Current Injection | Voltage Sensing |
|---|---|---|
| I | A - D | E - H |
| II | A - C | E - G |
| III | C - D | G - H |
| IV | B - C | F - G |
| V | B - D | F - H |
| VI | A - B | E - F |

The volunteer underwent a first set of 18 voltage measurements that included three replications of each voltage measurement for each vector. The first set of 18 measured voltage values was stored. Upon completion of these 18 measurements, the volunteer again voided his bladder, consumed a 20 ounce bottle of GATORADE sports drink fluid, and underwent a second set of 18 voltage measurements that included three replications for each of the six vectors. The second set of 18 measured voltage values was also stored. The variability of the three readings for each vector was small, demonstrating high consistency of the data. The coefficient of variation for all readings was 0.0078, indicating very low standard deviations relative to the means of each set of readings.

The changes in magnitude of electrical bio-impedance in ohms (delta zbar) computed from the first and second sets of measured voltages are summarized in Table 2.

TABLE 2

| delta zbar | I | % | II | % | III | % | IV | % | V | % | VI | % | rms (subj) |
|---|---|---|---|---|---|---|---|---|---|---|---|---|---|
| 1 | −15.48 | −11 | 4.58 | 3 | — | — | −0.04 | 0 | 13.65 | 8 | −3.43 | −3 | 9.576349 |
| 2 | −2.70 | −2 | −1.68 | −1 | — | — | −3.20 | −2 | 0.75 | 0 | −2.12 | −3 | 2.25271 |
| 3 | −95.26 | −120 | −2.97 | −1 | −18.13 | −6 | −9.89 | −4 | −11.02 | −6 | −8.07 | −10 | 40.20054 |
| 4 | −0.30 | 0 | 1.50 | 2 | — | — | −29.94 | −60 | 2.37 | 3 | 2.51 | 4 | 13.49349 |
| 5 | −3.03 | −3 | −2.25 | −2 | 1.84 | 2 | 0.24 | 0 | 2.11 | 2 | −5.62 | −9 | 2.990124 |
| 6 | 1.33 | 1 | −2.10 | −2 | 4.35 | 5 | −0.95 | −1 | 3.10 | 3 | −42.80 | −84 | 17.6418 |
| 7 | 11.59 | 14 | 0.00 | 0 | 0.30 | 1 | 1.73 | 2 | 6.82 | 8 | 2.06 | 4 | 5.599194 |
| 8 | 23.87 | 31 | 1.05 | 1 | 0.87 | 2 | 0.08 | 0 | −1.20 | −2 | −1.89 | −4 | 9.802272 |
| 9 | 1.80 | 2 | 1.65 | 2 | 1.64 | 3 | −0.18 | 0 | 1.86 | 2 | 1.44 | 2 | 1.541285 |
| 10 | −2.22 | −2 | −1.17 | −1 | 10.46 | 10 | 16.21 | 12 | 7.61 | 7 | −1.94 | −3 | 8.565593 |
| ms (lead) | 31.6931 | | 2.225782 | | 8.141708 | | 11.27332 | | 6.612787 | | 14.01664 | | |

The last column of Table 2 shows that the rms value of delta zbar of all six vectors for each volunteer exceeded the noise level (rms 3) for seven of the ten volunteers. These data demonstrate that in seven of the ten volunteers fluid uptake is correlated to electrical bio-impedance readings. (Because of the timing of GATORADE sports drink fluid intake and subsequent taking of measurements, only a very small amount of liquid could have escaped the volunteer's stomach or upper gastrointestinal tract.)

Table 2 shows only the magnitude component of the impedance measurements, for ease of presentation. A similar analysis was performed on the phase angle data collected from the readings. On this dataset the phase angle analysis provided somewhat greater sensitivity to fluid changes than the impedance magnitude alone. The phase angle data did not, however, materially affect the conclusions from the impedance magnitude data for each sample and for each vector.

A further two-step analysis on this dataset from the 10 volunteers compared the averages and ranges of the impedance measurements taken before and after fluid uptake. In the first step of the analysis, the average of the first, or pre-fluid-uptake, readings was compared to the range of the second, or post-fluid-uptake, readings. In the second step of the analysis, the average of the second, or post-fluid-uptake, readings was compared to the range of the first, or pre-fluid-uptake, readings. The purpose of this comparison was to determine how often the average of the first set of readings fell outside of the range of the second set of readings, and how often the average of the second set of readings fell outside of the range of the first set of readings. It was found that: 88% of the pre-fluid-uptake average readings fell outside the range of the post-fluid-uptake readings; 93% of the post-fluid-uptake average readings fell outside the range of the pre-fluid-uptake readings. This analysis further indicates that the data reliably show that impedance values significantly change with fluid uptake by the subject.

### EXAMPLE 2

The dataset set forth in Example 1 was compiled using the Xitron 4200 instrument, which uses multiple frequencies to inject current and to acquire and analyze the data. An additional study was undertaken with the RJL Physiological Event Analyzer (Model PEA) instrument, which uses a single (50 kHz) frequency to inject current and to acquire and analyze the data, to determine whether there are significant differences between the use of a single frequency and multiple frequencies. Paired readings, i.e., one from each instrument, were taken on a single human subject for each of the six vectors over the span of three hours at approximate 20-minute intervals as follows: three sets (a single measurement in each of all six vectors) of readings following micturation; one set of readings following intake of approximately eight ounces of GATORADE sports drink fluid; two sets of readings following an additional intake of approximately eight ounces of GATORADE sports drink fluid; and two sets of readings following micturation.

Analysis to determine the correlation between the two instruments was then performed to obtain a correlation coefficient for each of the six vectors. The average correlation coefficient for all vectors was 0.868. Table 3 presents the correlation coefficients for each vector.

TABLE 3

| Vector | Correlation Coefficient |
|---|---|
| I | 0.780 |
| II | 0.723 |
| III | 0.984 |
| IV | 0.936 |
| V | 0.900 |
| VI | 0.886 |

The data show a high correlation between the readings of the single-frequency instrument and the multiple-frequency instrument, indicating that either may be used for practicing the current invention.

It will be obvious to those having skill in the art that many changes may be made to the details of the above-described embodiment of this invention without departing from the underlying principles thereof. For example, analysis of an electrical bio-impedance value can be accomplished by chirp transform analysis or wavelet analysis. Further, variations on the algorithms developing the information used in identifying the presence of fluids within the body may incorporate data including other aspects of the sensed electrical signals, including phase angle. The scope of the present invention should, therefore, be determined only by the following claims.

1. A method of determining the presence of fluid volume in mammalian tissue of a mammal having a body, comprising:
providing a first set of injection electrodes and a second set of measurement electrodes;
positioning members of the first set of electrodes on the body to introduce electrical current flow through the mammalian tissue and thereby establish flow paths that define injection vectors along which electrical currents flow between two or more injection electrodes;
positioning members of the second set of electrodes on the body to define measurement vectors relating to electrical voltages produced in response to the electrical currents flowing between the injection electrodes, the injection and measurement vectors defining an anatomical space of the mammalian tissue;
deriving from each of different pairs of the injection and measurement vectors an electrical bio-impedance value that is characteristic of the electrical bio-impedance of a region of the anatomical space; and
analyzing the electrical bio-impedance values to detect a presence of a volume of fluid or change in a volume of fluid in the anatomical space.

2. The method of claim 1, in which the electrical current flow is introduced at multiple signal frequencies and the analyzing of the electrical bio-impedance value includes Fourier analysis and data reduction.

3. The method of claim 1, in which the electrical current flow is introduced by a complex electrical current waveform and the analyzing of the electrical bio-impedance value includes chirp transform analysis or waveform analysis.

4. The method of claim 1, in which the analyzing of the electrical bio-impedance values entails determining differences in the electrical bio-impedance values derived from the injection and measurement vectors.

5. The method of claim 4, further comprising determining temporal changes in the electrical bio-impedance values derived from the injection and measurement vectors.

6. The method of claim 1, in which the analyzing of the electrical bio-impedance values entails determining temporal changes in the electrical bio-impedance values derived from the injection and measurement vectors.

7. The method of claim 1, in which each member of the first set includes a current source and a current sink, the current source and current sink being positioned at locations on the body such that electrical current flowing from a current source of one of the members flows into a current sink of another one of the members.

8. The method of claim 1, in which each member of the first set includes multiple current sources and multiple current sinks, the current sources and current sinks being positioned at locations on the body such that electrical current flowing from a current source of one or electrical currents flowing from current sources of more than one of the members flow into one or more current sinks of another one of the members.

9. The method of claim 1, in which the injection and measurement vectors define a nominal shape of the anatomical space in the presence of a nominal quantity of fluid, and in which the presence of other than the nominal quantity of fluid changes the anatomical space from its nominal shape.

10. The method of claim 1, further comprising analyzing the electrical bio-impedance values to determine the extent of fluid volume in the mammalian tissue.

11. The method of claim 1, in which the fluid includes blood, and further comprising analyzing the electrical bio-impedance values to determine whether the presence of a volume of blood indicates an accumulation or a loss of blood.

12. An instrument for determining a presence of or change in fluid volume in mammalian tissue of a mammal having a body, comprising:
an injection current source of injection electrical current;
multiple electrodes configured for operative coupling to the injection current source and for placement on the body to introduce injection electrical current flow through the mammalian tissue and thereby establish flow paths that define injection vectors along which electrical currents flow between two or more injection electrodes;
multiple electrodes configured for placement on the body to define measurement vectors relating to electrical voltages produced in response to the electrical currents flowing between the injection electrodes;
sensor amplifier circuitry operatively coupled to the electrodes defining the measurement vectors to amplify the electrical voltages produced; and
processor circuitry operatively connected to the injection current source and the sensor amplifier circuitry, the processor circuitry programmed with instructions to process signals representing the injection electrical currents and the produced voltages corresponding to different pairs of the injection and measurement vectors, to compute from each of different pairs of the injection and measurement vectors an electrical bio-impedance value that characterizes the electrical bio-impedance of the mammalian tissue in an anatomical space, and to analyze the electrical bio-impedance values to detect a presence of or a change in a volume of fluid in the anatomical space.

13. The instrument of claim 12, further comprising memory stores operatively associated with the processor circuitry to store the computed electrical bio-impedance values, the memory stores being separable from the instrument and capable of retaining the computed electrical bio-impedance values upon separation from the instrument.

14. The instrument of claim 12, further comprising an internal electrical power supply, thereby facilitating instrument portability.

15. The instrument of claim 12, in which the sensor amplifier circuitry includes amplifier circuitry operating in a differential input mode and having a gain value suitable for electrocardiogram (ECG) signal acquisition, the amplifier circuitry operating in a differential input mode having an output, and in which selected ones of the multiple electrodes provide electrical voltages representing acquired ECG signals, multiple ones of the acquired ECG signals being operatively coupled to the amplifier circuitry operating in a differential input mode, and further comprising:
pulse-generator pulse amplifier and detector circuitry to which an analog signal produced across two of the multiple electrodes is operatively coupled, the pulse-generator pulse amplifier and detector circuitry producing an output in response to characteristics of the analog signal that represent signal characteristics of a pulse produced by a pulse generator operatively connected to the mammal; and
the processor circuitry programmed with instructions to process signals corresponding to the outputs of the amplifier circuitry operating in the differential input mode and the pulse-generator pulse amplifier and detector circuitry to produce an electrocardiogram signal representation.

16. The instrument of claim 15, in which the electrocardiogram signal representation produced includes a signal component that indicates a presence or an absence of a pulse-generator pulse.

17. The instrument of claim 12, in which:
the sensor amplifier circuitry includes amplifier circuitry operating in a differential input mode and having a gain value suitable for electrocardiogram (ECG) signal acquisition, the amplifier circuitry operating in a differential input mode having an output;
selected ones of the multiple electrodes provide electrical voltages representing acquired ECG signals, multiple ones of the acquired ECG signals being operatively coupled to the amplifier circuitry operating in a differential input mode; and
the processor circuitry is programmed with instructions to process signals corresponding to the output of the amplifier circuitry operating in the differential input mode to produce an electrocardiogram signal representation.

18. The instrument of claim 12, further comprising electrode selector switch circuitry through which the multiple electrodes are operatively coupled to the injection current source and the sensor amplifier circuitry, the electrode selector switch circuitry responsive to command information delivered from the processor circuitry to select which ones of the multiple electrodes introduce the electrical current flow and which ones of the multiple electrodes define measurement vectors relating to the electrical voltages produced.

19. The instrument of claim 18, in which the electrode selector switch circuitry is configured for independent selection of the multiple electrodes in response to the command information.

20. The instrument of claim 18, in which the command information delivered to the electrode selector switch circuitry selects sets of the multiple electrodes to define multiple electrode assemblies, each of the multiple electrode assemblies including on a common substrate a first electrode structure that introduces injection electrical current flow and a second electrode structure that defines a measurement vector relating to the electrical voltages produced.

21. The instrument of claim 20, in which each of the first and second electrode structures includes at least one electrode segment.

22. The instrument of claim 12, further comprising a housing connector to which a connection block module is releasably attachable for matable connection, the multiple electrodes connected by associated electrically conductive leads to the connection block module, and the connection block module including at least one of a battery, defibrillator discharge protection, or memory.

23. The instrument of claim 12, further comprising an enclosure in which the instrument is contained, the enclosure further containing one or more equipment modules that form with the instrument an integrated system by common operational access to one or more of a display, power supply, memory, controls, or input/output connection.

24. The instrument of claim 12, further comprising an enclosure in which the instrument is contained, the enclosure further containing a collection of one or more independently operating equipment modules.

25. The instrument of claim 12, further comprising an input/output connection device that is operatively associated with the processor circuitry and is configured to receive information from and to export information to an external location.

26. The instrument of claim 25, in which the input/output device and the external location are operatively connected by a communication link of one of a wire line or a wireless medium type.
